(19) United States
(12) Patent Application Publication
(10) Pub. No.: US 2009/0209925 A1
(43) Pub. Date: Aug. 20, 2009

(54) DEVICE FOR THE TREATMENT OF CAROTID ARTERY DISEASES

(76) Inventors: Andrea Marinello, Verona (IT); Vittorio Marinello, Verona (IT)

Correspondence Address: LADAS & PARRY LLP, 224 SOUTH MICHIGAN AVENUE, SUITE 1600, CHICAGO, IL, 60604, US

(21) Appl. No.: 12/159,248

(22) PCT Filed: Dec. 28, 2005
(86) PCT No.: PCT/IB2005/003900

§ 371 Date: Nov. 17, 2008

(51) Int. Cl. A61M 35/00; A61F 13/12

(52) U.S. Cl. 604/308

(57) ABSTRACT

Device comprising a collar, at least one inflatable elastic cushion, and a pad, carried by the cushion and comprising a revulsive active principle. Also envisaged is the use of a revulsive active principle for the preparation of a medicament for treatment of an affection of the carotid arteries.

### TECHNICAL FIELD

The present invention relates to a device in particular for treatment of an affection of the carotid arteries.

### BACKGROUND ART

Solid emboli, generally constituted by aggregates of blood cells, are amongst the main causes of partial or total obstruction of blood vessels.

Following upon a lesion of a blood vessel or the presence of high values of lipids in the blood, there may arise formation of lipid plaques and/or plaques formed by blood cells, referred to as atheromatous plaques, which can reduce the calibre of the vessels even to a marked extent and hence reduce irrigation by the blood of the tissues in which said vessels are located.

It may occur, on occasion, that portions may detach from said plaques to form the so-called emboli, which circulate freely in the blood stream.

When such a portion detaches from an atheromatous plaque present at the level of the heart or carotid, the embolus thus formed may reach the central nervous system and occlude a capillary of the brain.

The above occlusion causes the lack of irrigation of a more or less extensive area of the brain, which in some cases may cause cerebral embolism.

Numerous forms of treatment are known both of an invasive type and of a non-invasive type for treating and/or preventing such affections, which so far, however, have not afforded a solution to the problem.

It is known that the formation of atheromatous plaques is often accompanied by inflammatory phenomena of the tissue surrounding the blood vessel concerned.

The inflammation involves various steps that lead to a morphological alteration of the inflamed tissue. In particular, the restriction of the vessel lumen may initially be noted, which is then followed by a dilation thereof, accompanied by hyperaemia. Said behaviour is referred to as vessel reaction.

Following upon inflammation, mobile phagocytes are recalled by the affected tissue, which, together with the local histocytes, ingest the lipids that deposit along the vessel walls.

Should, however, the activity of the leukocytes in the blood be insufficient to eliminate completely infiltration of lipids in the vascular intima, the inflammation becomes chronic.

A known treatment of chronic inflammatory phenomena is by thermal and inhalational therapy, hydrotherapy, mud-baths, and flow of steam capable of bringing about a dilation of the blood vessels.

However, such forms of treatment have proven ineffective in combating deep internal inflammatory phenomena.

Research is consequently directed at identifying a treatment, as well as a device for administration thereof, that will be able to prevent or treat inflammation of vascular tissues, in particular at a carotid level, the formation of atheromatous plaques and hence reduce the risk of cerebral embolism. In addition, it would be desirable to identify a treatment, as well as a device for administration thereof, that can be successfully applied to a wide range of patients and that presents a low cost and will thus enable extension of application of said treatment also to health systems with limited economic resources.

### DISCLOSURE OF INVENTION

The aim of the present invention is to provide a treatment for an affection of the carotid arteries, in particular for cerebral embolism, as well as a device for administration thereof, that will be free from the disadvantages of the known art.

In particular, according to the present invention, said aim is achieved by means of a device that comprises a collar, at least one inflatable elastic cushion carried by said collar, and a pad carried by said inflatable cushion and comprising a revulsive active principle, inflation of said cushion facilitating absorption of the active principle.

The active principle is preferably a dried herb, more preferably a crucifer, moistened with water.

Preferably, the crucifers are selected from the group consisting of mustard, radish, and cress, which all contain, in different quantities, allyl isothiocyanate (C₃H₅NCS), which is a substance that is able to elicit a vasomotor reaction.

It is known that allyl isothiocyanate, which is present in high concentrations in various plants, can cause vesicatory phenomena, in particular when they are applied in the proximity of sensitive tissues, for example the mucosae. Consequently, its use at high concentrations is certainly not recommended.

According to the present invention, the dried herbs are used after being moistened with water, preferably at a temperature of 10° C.

In this way, the allyl isothiocyanate present therein is diluted, thus reducing its harmful effects, but preserving its vasomotor properties.

In order to reach deep internal vessels, such as the primitive and internal carotids, the device according to the invention is provided with at least one inflatable elastic cushion, inflation of which promotes the in-depth absorption of the revulsive active principle.

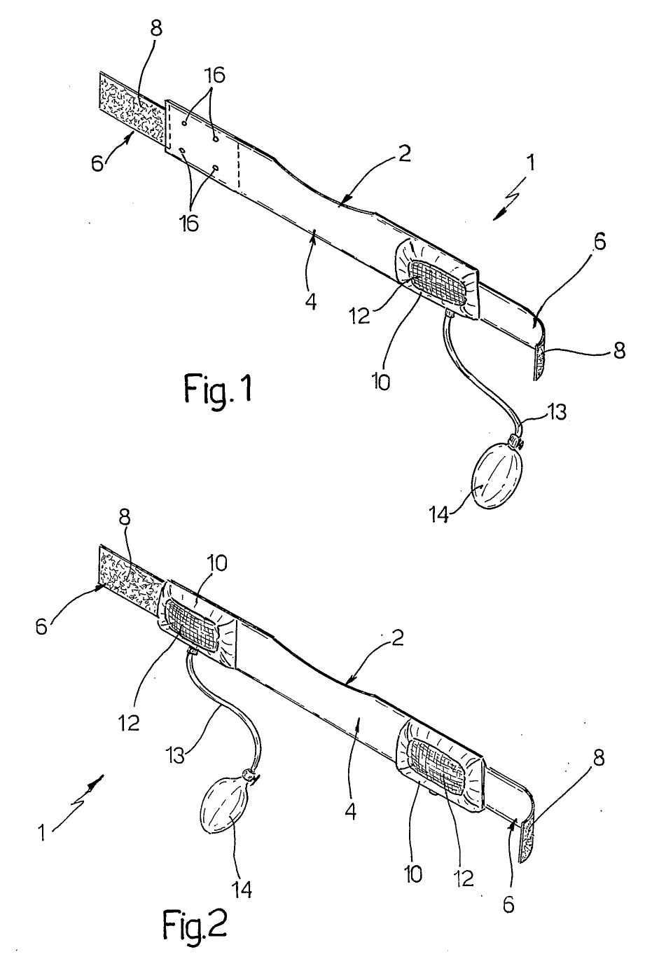

Further characteristics and advantages of the device according to the present invention will emerge clearly from the ensuing description of a non-limiting example of embodiment thereof, described with reference to the annexed plate of drawings, wherein FIGS. 1, 2 and 3 illustrate, in perspective view, the device according the present invention set in different operative configurations.

### BRIEF DESCRIPTION OF THE DRAWINGS

Designated, as a whole, by 1 in FIGS. 1, 2 and 3 is a device made according to the teachings of the present invention and comprising:
a collar 2 (of a known type), which comprises a flexible rectangular body 4 (for example, made of cotton) provided, at its end, with an adjustable fastening device 6, formed by a pair of strips 8 provided with Velcro™;
a pair of inflatable elastic cushions 10 (of a known type) spaced out with respect to one another along the body 4; and
a pair of pads 12 (made of absorbent material, for example cotton), each carried by a respective cushion 10 and comprising a revulsive active principle.

In particular, each cushion 10 defines within it a compression chamber, which communicates, through a flexible tube 13, with an inflation device of a manual type 14, for example a deformable bladder, which, when subjected to an external manual pressure, compresses the air contained therein and sends it to the cushion 10.

In addition, a connection device of a snap-action type 16 (for example, comprising four press-studs—illustrated only in FIG. 1) is set between the body 4 and the cushion 10 to enable removal of the cushion 10 for cleaning and/or replacement thereof.

Also provided is a connection device of a reversible type, for example, of the Velcro™ type (not illustrated), to enable a stable positioning of the pad 12 on the outer surface of the cushion 10.

The device according to the present invention is designed, in use, to be fixed to the patient's neck by means of the pair of strips 8 provided with Velcro™ at their ends.

The spacing between the two inflatable cushions 10 is such as to enable their positioning against the right and left carotid arteries.

A pad 12, soaked in the revulsive active principle is applied on each cushion 10, using Velcro™.

After the inflatable cushions 10 have been inflated using the manual-inflation device 14, absorption of the revulsant through the skin is promoted.

The device according to the invention, by enabling release of the revulsive active principle, is suited to the treatment of affections of the carotid arteries, in particular of carotid restriction, atherosclerosis, and of cerebral embolism.

The present invention moreover proposes the use of a revulsive active principle for the preparation of a medicament for the treatment of an affection of the carotid arteries, preferably selected in the group consisting of carotid restriction, atherosclerosis, and cerebral embolism.

Preferably, the revulsive active principle is a dried herb moistened with water, more preferably a crucifer.

Preferably, the dried herb is selected in the group consisting of mustard, radish, and cress, which are herbs rich in allyl isothiocyanate (C₃H₅NCS).

Preferably, the revulsive dried herb is moistened with water at a temperature of 10° C.

In a preferred embodiment of the invention, the medicament is prepared in the form of a transdermal plaster or alternatively is released using a device according to the present invention.

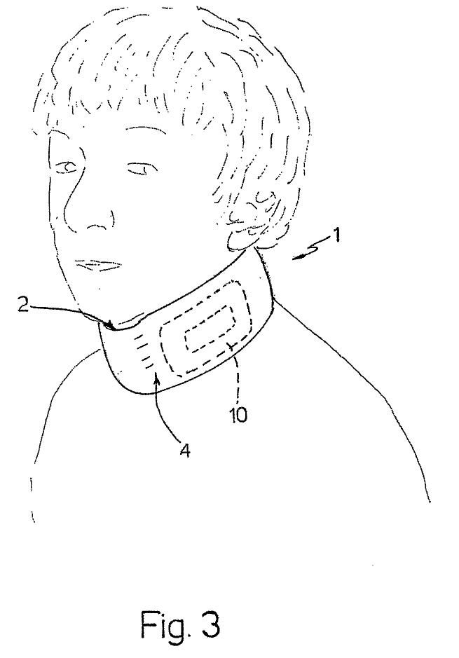

In use, the plaster is applied to the skin in such a way that the revulsive active principle is released. In addition, in order to promote administration of the active principle, a collar 2 (illustrated in FIG. 3) provided with at least one inflatable elastic cushion 10 carried by said collar 2 is set on top said plaster and is fixed to the body of the patient. Subsequent inflation of the inflatable cushion 10 (obtained, as described above, by acting on the inflation device 14) concurs in applying a localized pressure on said plaster in order to facilitate absorption of the active principle through the skin.

Once the active principle has been administered via transdermal route, it reaches the blood vessels of the carotid arteries, so causing hyperaemia, with consequent mobilization of mobile phagocytes, and thus promoting the vasomotor reaction.

1. A device comprising a collar, at least one inflatable elastic cushion carried by said collar, and a pad carried by said inflatable cushion and comprising a revulsive active principle, inflation of said cushion facilitating absorption of the active principle.

2. The device according to claim 1, characterized in that said active principle is a dried herb moistened with water.

3. The device according to claim 2, characterized in that said dried herb is a crucifer.

4. The device according to claim 2, characterized in that said dried herb is selected in the group consisting of mustard, radish and cress.

5. The device according to claim 2, characterized in that said dried herb contains allyl isothiocyanate (C₃H₅NCS).

6. The device according to claim 2, characterized in that said water is at a temperature of 10° C.

7. The device according to claim 1, characterized in that connection means of a releasable type are provided, set between said cushion and said collar.

8. The device according to claim 7, in which said connection means of a releasable type comprise snap-action connection means.

9. The device according to claim 1, characterized in that said collar is provided at its ends with fastening means of an adjustable type, in particular of a Velcro™ type.

10. The device according to claim 1, characterized in that said inflatable elastic cushion comprises an internal compression chamber communicating, through an elastic tube, with manually operable inflation means.

11. The device according to claim 1, characterized in that said pad is positioned along said collar in such a way that it is set, in use, in contact with the part of the neck where the carotid arteries pass.

12. The device according to claim 1, characterized in that said collar comprises two inflatable elastic cushions supporting respective pads, said pads being positioned along said collar in such a way that each pad is set, in use, in contact with the part of the neck where the left and right carotid arteries pass.

13. A kit for treatment of carotid affections including a transdermal plaster, which comprises a revulsive active principle and a collar provided with at least one inflatable elastic cushion, carried by said collar, inflation of said cushion acting on said plaster in order to facilitate absorption of the active principle.

14. Use of a revulsive active principle for the preparation of a medicament for the treatment of an affection of the carotid arteries.

15. Use according to claim 14, characterized in that said affection is selected in the group consisting of carotid restriction, atherosclerosis and cerebral embolism.

16. Use according to claim 14, characterized in that said revulsive active principle is a dried herb moistened with water.

17. Use according to claim 16, characterized in that said dried herb is a crucifer.

18. Use according to claim 16, characterized in that said dried herb is selected in the group consisting of mustard, radish and cress.

19. Use according to claim 16, characterized in that said dried herb contains allyl isothiocyanate (C₃H₅NCS).

20. Use according to claim 16, characterized in that said water is at a temperature of 10° C.

21. Use according to claim 14 via transdermal route.

22. Use according to claim 14, characterized in that said medicament is a transdermal plaster.
